(19) United States
(12) Patent Application Publication
(10) Pub. No.: US 2003/0013673 A1
(43) Pub. Date: Jan. 16, 2003

(54) Modulating the Rad-nm23 interaction

(76) Inventors: C. Ronald Kahn, West Newton, MA (US); Jianhua Zhu, Bowling Green, KY (US)

Correspondence Address: LOUIS MYERS, Fish & Richardson P.C., 225 Franklin Street, Boston, MA, 02110-2804, US

(21) Appl. No.: 10/074,694

(22) Filed: Feb. 12, 2002

Related U.S. Application Data
(63) Continuation of application No. 09/053,967, filed on Apr. 2, 1998, now abandoned.
(60) Provisional application No. 60/043,983, filed on Apr. 3, 1997.

(51) Int. Cl. A61K 48/00; A61K 31/00

(52) U.S. Cl. 514/44; 514/1

(57) ABSTRACT

The present invention is based on the discovery of a novel bimolecular, bidirectional mechanism of regulation between nm23 and Rad. Accordingly, the present invention provides a method of modulating an activity of nm23 in a subject at risk for a proliferative disorder, comprising: modulating the level of Rad activity.

### RELATED APPLICATIONS

This application claims priority to U.S. provisional Application No. 60/043,983, filed on Apr. 3, 1997, the contents of which are incorporated herein by reference.

### BACKGROUND OF THE INVENTION

The invention relates to the interaction of Rad and nm23.

### SUMMARY OF THE INVENTION

The inventors have discovered a novel bimolecular, bidirectional mechanism of regulation between nm23 and Rad. nm23 regulates Rad's state of activity by altering the balance of GTP and GTP loading, while Rad alters the state of nm23 activity as an NDP kinase.

In general, the invention features, a method of modulating an activity of nm23 in a cell or a subject, e.g., a human at risk for a disorder characterized by unwanted cell proliferation. The method includes: modulating the level of Rad activity, e.g., by modulating the level of Rad expression, to thereby modulate nm23 activity. The level of Rad activity can be increased, e.g., by administering a Rad polypeptide, or a Rad polypeptide encoding nucleic acid, to the cell or subject. The level can be decreased, e.g., by administering an antisense nucleic acid which decreases Rad expression, or by administering an anti-Rad antibody, or a nucleic acid which encodes an anti-Rad antibody, preferably an “intra-body”. The level of Rad can also be decreased by administering a dominant negative Rad mutant polypeptide or a nucleic acid which encodes such a mutant polypeptide. Increasing Rad activity increases nm23 activity. Decreasing Rad activity decreases nm23 activity.

While not wishing to be bound by theory, the inventors believe that Rad can modulate nm23 activity by one or both of increasing the nucleoside diphosphate kinase activity of nm23 or by promoting the autophosphorylation of nm23.

In another aspect, the invention features a method of modulating the activity of Rad in a cell or a subject, e.g., a human at risk for an insulin-related disorder. The method includes: modulating the level of nm23 activity, e.g., by modulating the level of nm23 expression, to thereby modulate Rad activity. The level of nm23 activity can be increased, e.g., by administering a nm23 polypeptide, or a nm23 polypeptide encoding nucleic acid, to the cell or subject. The level can be decreased, e.g., by administering an antisense nucleic acid which decreases nm23 expression, or by administering an anti-nm23 antibody, or a nucleic acid which encodes an anti-nm23 antibody, preferably an “intra-body”. The level of nm23 can also be decreased by administering a dominant negative nm23 mutant polypeptide or a nucleic acid which encodes such a mutant polypeptide Increasing nm23 activity increases Rad activity. Decreasing nm23 activity decreases Rad activity.

In another aspect, the invention features, a method of modulating the developmental state of a cell, e.g., a neural or muscle cell, or a subject by modulating the level of Rad activity. The method includes: modulating the level of Rad activity, e.g., by modulating the level of Rad expression. The level of Rad activity can be increased, e.g., by administering a Rad polypeptide, or a Rad polypeptide encoding nucleic acid, to the cell or subject. The level can be decreased, e.g., by administering an antisense nucleic acid which decreases Rad expression, or by administering an anti-Rad antibody, or a nucleic acid which encodes an anti-Rad antibody, preferably an “intra-body”. The level of Rad can also be decreased by administering a dominant negative Rad mutant polypeptide or a nucleic acid which encodes such a mutant polypeptide. In preferred embodiments increasing Rad activity delays cell cycle transition and promotes differentiation of the cell; decreasing Rad activity has the opposite effect.

In preferred embodiments the cell is a cultured cell.

In preferred embodiments the developmental state is modulated to promote wound healing or tissue replacement.

In another aspect, the invention features, a method of modulating apoptosis in a cell, e.g., a cancer cell, or a subject, by modulating the level of Rad activity. The method includes: modulating the level of Rad activity, e.g., by modulating the level of Rad expression. The level of Rad activity can be increased, e.g., by administering a Rad polypeptide, or a Rad polypeptide encoding nucleic acid, or subject. The level can be decreased, e.g., by administering an antisense nucleic acid which decreases Rad expression, or by administering an anti-Rad antibody, or a nucleic acid which encodes an anti-Rad antibody, preferably an “intra-body”. The level of Rad can also be decreased by administering a dominant negative Rad mutant polypeptide or a nucleic acid which encodes such a mutant polypeptide. In preferred embodiments increasing Rad activity increases apoptosis; decreasing Rad activity inhibits apoptosis.

In another aspect, the invention features, treating a disorder characterized by unwanted cell proliferation or unwanted cell migration, in a subject. The method includes: increasing the level of Rad activity, e.g., by modulating the level of Rad expression. The level of Rad activity can be increased, e.g., by administering a Rad polypeptide, or a Rad polypeptide encoding nucleic acid, to the subject.

In another aspect, the invention features, a method of, modulating the mobility or motility of a cell. The method includes: modulating the level of Rad activity, e.g., by modulating the level of Rad expression. The level of Rad activity can be increased, e.g., by administering a Rad polypeptide, or a Rad polypeptide encoding nucleic acid, to the cell or subject. The level can be decreased, e.g., by administering an antisense nucleic acid which decreases Rad expression, or by administering an anti-Rad antibody, or a nucleic acid which encodes an anti-Rad antibody, preferably an “intra-body”. The level of Rad can also be decreased by administering a dominant negative Rad mutant polypeptide or a nucleic acid which encodes such a mutant polypeptide. Increasing Rad activity decreases mobility or motility. Decreasing Rad activity increases mobility or motility.

In another aspect, the invention features a method of treating a subject at risk for an insulin-related disorder. The method includes: modulating the level of nm23 activity, thereby treating the disorder. Although not wishing to be bound by theory the inventors believe that modulating the level of nm23 activity modulates the level of Rad activity. Therefore, the method includes increasing the level of nm2 activity in disorders where the level of Rad activity is too low and decreasing the level of mn2 activity in disorders where the level of Rad activity is too high. The level of nm23 activity can be increased, e.g., by administering a nm23 polypeptide, or a nm23 polypeptide encoding nucleic acid, to the subject. The level can be decreased, e.g., by administering an antisense nucleic acid which decreases nm2 expression, or by administering an anti- nm23 antibody, or a nucleic acid which encodes an anti- nm23 antibody, preferably an “intra-body”. The level of nm23 can also be decreased by administering a dominant negative Rad mutant polypeptide or a nucleic acid which encodes such a mutant polypeptide.

In another aspect, the invention features, a method of evaluating the metastatic potential of a tissue, e.g., a tumor from a subject. The method includes: providing a tissue sample; and performing one or mare of the following, evaluating the level of Rad expression, evaluating the level of Rad activity, or determining if there is a lesion in the Rad gene or in the Rad control region. Lower than wild type level of expression or activity, or non-wildtype genomic structure, is correlated with metastatic potential.

In another aspect, the invention provides, a method of determining if a subject mammal, e.g., a primate, e.g., a human, is at risk for a disorder related to unwanted cell proliferation, and in preferred embodiments, risk for metastasis. The method includes detecting, in a tissue of the subject, the presence or absence of a mutation of a Rad gene, or a homolog thereof. In preferred embodiments: detecting the mutation includes ascertaining the existence of at least one of: a deletion of one or more nucleotides from the gene; an insertion of one or more nucleotides into the gene, a point mutation, e.g., a substitution of one or more nucleotides of the gene, a gross chromosomal rearrangement of the gene, e.g., a translocation, inversion, or deletion.

In another aspect, the invention provides, a method of determining if a subject mammal, e.g., a primate, e.g., a human, is at risk for a disorder related to unwanted cell proliferation, and in preferred embodiments risk for metastasis. The method includes detecting, in a tissue of the subject, a non-wild type level of a Rad RNA or polypeptide.

In another aspect, the invention provides, a method of determining if a subject mammal, e.g., a primate, e.g., a human, is at risk for a disorder related to unwanted cell proliferation, and in preferred embodiments risk for metastasis. The method includes detecting, in a tissue of the subject, the mis-expression of a gene encoding a Rad protein. In preferred embodiments: detecting the misexpression includes ascertaining the existence of at least one of: an alteration in the level of a messenger RNA transcript of the gene; the presence of a non-wild type splicing pattern of a messenger RNA transcript of the gene; or a non-wild type level of the protein.

In another aspect, the invention provides, a method of determining if a subject mammal, e.g., a primate, e.g., a human, is at risk for an insulin-related disorder. The method includes detecting, in a tissue of the subject, the presence or absence of a mutation of a nm23 gene, or a homolog thereof. In preferred embodiments: detecting the mutation includes ascertaining the existence of at least one of: a deletion of one or more nucleotides from the gene; an insertion of one or more nucleotides into the gene, a point mutation, e.g., a substitution of one or more nucleotides of the gene, a gross chromosomal rearrangement of the gene, e.g., a translocation, inversion, or deletion.

In another aspect, the invention provides, a method of determining if a subject mammal, e.g., a primate, e.g., a human, is at risk for an insulin-related disorder. The method includes detecting, in a tissue of the subject, a non-wild type level of a nm23 RNA or polypeptide.

In another aspect, the invention provides, a method of determining if a subject mammal, e.g., a primate, e.g., a human, is at risk for an insulin-related disorder. The method includes detecting, in a tissue of the subject, the mis-expression of a gene encoding a nm23 protein. In preferred embodiments: detecting the misexpression includes ascertaining the existence of at least one of: an alteration in the level of a messenger RNA transcript of the gene; the presence of a non-wild type splicing pattern of a messenger RNA transcript of the gene; or a non-wild type level of the protein.

Diagnostic methods disclosed herein can be performed prenatally, on infants, children, or adults.

In another aspect, the invention features, a method of evaluating a fragment or analog of the Rad protein for the ability to interact with nm23. The interaction can be any interaction between the Rad polypeptide and the nm23 polypeptide, e.g., binding of the Rad and nm23 polypeptide, alteration of the three dimensional structure of the Rad or nm23 polypeptide, or covalent or non-covalent modification of the Rad or nm23 polypeptide, e.g., the phosphorylation or autophosphylation of the nm 23 polypeptides. The method includes: contacting the Rad polypeptide with the nm23 polypeptide; and evaluating the ability of the two to interact. This method can be performed in vitro, e.g., in a cell free system, or in vivo, e.g., in a two-hybrid interaction trap assay.

The method is particularly useful for identifying fragments or analogs of Rad which have biological activity or which can bind nm23. This method is useful for identifying fragments or analogs which are agonists or antagonists of Rad.

In another aspect, the invention features, a method of evaluating a fragment or analog of the nm23 protein for the ability to interact with Rad. The interaction can be any interaction between the Rad polypeptide and the nm23 polypeptide, e.g., binding of the Rad and nm23 polypeptide, alteration of the three dimensional structure of the Rad or mn23 polypeptide, or covalent or non-covalent modification of the Rad or nm23 polypeptide, e.g., the phosphorylation or autophosphylation of the nm 23 polypeptides. The method includes: contacting the Rad polypeptide with the nm23 polypeptide; and evaluating the ability of the two to interact. This method can be performed in vitro, e.g., in a cell free system, or in vivo, e.g., in a two-hybrid interaction trap assay.

The method is particularly useful for identifying fragments or analogs of nm23 which have biological activity or which can bind Rad. This method is useful for identifying fragments or analogs which are agonists or antagonists of nm23.

In another aspect, the invention features a method for evaluating a test compound, e.g., an inhibitor candidate, to modulate an interaction of a Rad polypeptide with a nm23 polypeptide. The method includes the steps of:

(i) providing a reaction mixture which includes the test compound, a Rad polypeptide, and a nm23 polypeptide, preferably under conditions wherein in the absence of the test compound, the Rad polypeptide and the mn23 polypeptide, interact; and

(ii) detecting an interaction between the Rad and nm23 polypeptides , e.g., detecting the formation (or dissolution) of a complex between the Rad and nm23 polypeptides. A change, e.g., a decrease or increase, in the formation of the complex in the presence of the test compound (relative to what is seen in the absence of the compound) is indicative of a modulation.

The test compound can be e.g., an inhibitor candidate, e.g., a fragment or analog of Rad or nm23, or a small molecule mimic thereof

The interaction can be any interaction between the Rad and nm23 peptides, e.g., binding of the polypeptides, alteration of the three dimensional structure of either polypeptide, or covalent or non-covalent modification of either polypeptide, e.g., the phosphorylation or autophosphylation of the nm23 polypeptides.

In preferred embodiments one or more of the Rad polypeptide, the nm23 polypeptide, or the compound is a purified preparation.

The method can be performed a cell-free system, e.g., a cell lysate or a reconstituted protein mixture. The polypeptides, and the compound, can be expressed in a cell, and the cell contacted with the first compound, e.g. in an interaction trap assay (e.g., a two-hybrid assay).

In yet another aspect, the invention features a two-phase method (e.g., a method having an in vitro, e.g., a cell free system, and an in vivo phase) for evaluating a compound, for the ability to modulate an interaction of a Rad and a nm23 polypeptide.

The method includes steps (i) and (ii) of the method described immediately above performed in vitro, and further includes: (iii) determining if the compound modulates the interaction in vitro, e.g., in a cell free system, and if so; (iv) administering the compound to a cell or animal; and optionally,(v) evaluating the in vivo effect of the compound on an interaction of Rad and nm23, e.g., by the effect on cell growth, or by the effect on the expression of a reporter gene.

In another aspect, the invention features a two-phase method (e.g., a method having a primary in vitro and a secondary in vivo phase) for evaluating a treatment. The method can be used to evaluate a treatment for the ability to modulate, e.g., to inhibit or promote, a Rad-nm23 interaction, or to evaluate test compounds for use as therapeutic agents. The method includes: (i) an in vitro phase in which the test compound is contacted with a cell, or a cell free system, which includes a reporter gene functionally linked to a Rad or nm23 regulatory sequence, and detecting the modulation of the expression of the reporter gene and (ii) if the test compound modulates the expression, administering the test compound to an animal, and evaluating the in vivo effects of the compound on a parameter related to intracellular signaling, e.g., lymphocyte activation or proliferation.

In another aspect, the invention features a cell or purified preparation of cells which include a Rad and an nm23 transgene, or which otherwise misexpresses a Rad and an nm23 gene. The cell preparation can consist of human or non human cells, e.g., rodent cells, e.g., mouse or rat cells, rabbit cells, or pig cells. In preferred embodiments, the cell or cells include a Rad and an nm23 transgene, e.g., a heterologous form of such gene, e.g., a gene derived from humans (in the case of a non-human cell). The transgenes can be misexpressed, e.g., overexpressed or underexpressed. In other preferred embodiments, the cell or cells include a gene which misexpress an endogenous Rad and nm23 genes, e.g., a gene the expression of which is disrupted, e.g., a knockout. Such cells can serve as a model for studying disorders which are related to mutated or mis-expressed alleles or for use in drug screening.

In another aspect, the invention features a transgenic Rad-nm23 animal, e.g., a rodent, e.g., a mouse or a rat, a rabbit, or a pig. In preferred embodiments, the transgenic animal includes (and preferably express) a heterologous form of a Rad or nm23 gene, e.g., a gene derived from humans. In other preferred embodiments, the animal has an endogenous Rad or nm23 gene which is misexpressed, e.g., a knockout. Such a transgenic animal can serve as a model for studying disorders which are related to mutated or mis-expressed alleles or for use in drug screening.

In another aspect, the invention features a method of evaluating an effect of a treatment, e.g., a treatment used to treat a disorder characterized by aberrant or unwanted cell proliferation. The method uses a wild type test cell or organism, or a cell or organism which misexpresses a Rad and an nm23 encoding sequence or which has a Rad and an mn23 transgene. The method includes: administering the treatment to a test cell or organism, e.g., a cultured cell, or a mammal, and evaluating the effect of the treatment on a parameter related to, e.g., cell proliferation. An effect on the parameter indicates an effect of the treatment.

An insulin-related disorder, as used herein, includes diabetes, e.g., type II diabetes, obesity, or any disorders involving insulin-stimulated glucose transport, and disorders involving abnormally low or abnormally high levels of Rad activity or expression.

A disorder characterized by unwanted cell proliferation as, used herein, includes, by way of example, cancer. Cancers which can be diagnosed or treated by methods disclosed herein include cancers with metastatic potential, melanoma, breast carcinoma, hepatocellular carcinoma, leukemia, colon and colorectal carcinomas, and ovarian carcinoma. Treatment can result in one or more of reduced incidence of primary tumor formation, decreased metastasis, and decreased response to growth factors.

A subject, as used herein, refers to a mammal, e.g., a human. It can also refer to an experimental animal, e.g., an animal model for an insulin-related disorder, e.g., a NOD mouse, an ob/ob mouse, a db/db mouse, a Zucker fatty rat, or a streptozotocin induced mouse or rat. The subject can be a human which is at risk for a disorder characterized by unwanted cell proliferation. The subject can also be a human at risk for an insulin-related disorder. “nm23”, as used herein, refers to any member of the nm23 family, e.g., H1 or H2.

A “heterologous promoter”, as used herein is a promoter which is not naturally associated with a gene or a purified nucleic acid.

A “purified preparation” or a “substantially pure preparation” of a polypeptide, as used herein, means, in the case of a naturally occurring polypeptide, a polypeptide that has been separated from at least one other protein, lipid, or nucleic acids with which it naturally occurs. In the case of either a naturally occurring or non-naturally occurring polypeptide, the polypeptide is preferably separated from substances, e.g., antibodies or gel matrix, e.g., polyacrylamide, which are used to purify it. In the case of either a naturally occurring or non-naturally occurring polypeptide, the polypeptide is preferably constitutes at least 10, 20, 50 70, 80 or 95% dry weight of the purified preparation. Preferably, the preparation contains: sufficient polypeptide to allow protein sequencing; at least 1, 10, or 100,mg of the polypeptide; at least 1, 10, or 100 mg of the polypeptide.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

A “treatment”, as used herein, includes any therapeutic treatment, e.g., the administration of a therapeutic agent or substance, e.g., a drug.

The “metabolism of a substance”, as used herein, means any aspect of the, expression, function, action, or regulation of the substance. The metabolism of a substance includes modifications, e.g., covalent or non covalent modifications of the substance. The metabolism of a substance includes modifications, e.g., covalent or non covalent modification, the substance induces in other substances. The metabolism of a substance also includes changes in the distribution of the substance. The metabolism of a substance includes changes the substance induces in the structure or distribution of other substances.

A “substantially pure nucleic acid”, e.g., a substantially pure DNA, is a nucleic acid which is one or both of: not immediately contiguous with either one or both of the sequences, e.g., coding sequences, with which it is immediately contiguous (i.e., one at the 5′ end and one at the 3′ end) in the naturally-occurring genome of the organism from which the nucleic acid is derived; or which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences.

“Homology, or sequence identity”, as used herein, refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or have sequence identity at that position. The percent of homology or sequence identity between two sequences is a function of the number of matching or homologous positions shared by the two sequences divided by the number of positions compared ×100. For example, if 6 of 10, of the positions in two sequences are matched or homologous then the two sequences are 60% homologous or have 60% sequence identity. By way of example, the DNA sequences ATTGCC and TATGGC share 50% homology or sequence identity. Generally, a comparison is made when two sequences are aligned to give maximum homology or sequence identity.

The comparison of sequences and determination of percent homology between two sequences can also be accomplished using a mathematical algorithim. A preferred, non-limiting example of a mathematical algorithim utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can also be performed with the XBLAST program, score=50, wordlength=3 to obtain amino acid sequences homologous to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithim utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The terms “peptides”, “proteins”, and “polypeptides” are used interchangeably herein.

As used herein, the term “transgene” means a nucleic acid sequence (encoding, e.g., one or more Rad or nm23 polypeptides), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of the selected nucleic acid, all operably linked to the selected nucleic acid, and may include an enhancer sequence.

As used herein, the term “transgenic cell” refers to a cell containing a transgene.

As used herein, a “transgenic animal” is any animal in which one or more, and preferably essentially all, of the cells of the animal includes a transgene. The transgene can be introduced into the cell, directly or indirectly by introduction into a precursor of the cell, by way of deliberate genetic manipulation, such as by microinjection or by infection with a recombinant virus. This molecule may be integrated within a chromosome, or it may be extrachromosomally replicating DNA.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue. The term also covers so-called “leaky” promoters, which regulate expression of a selected DNA primarily in one tissue, but cause expression in other tissues as well.

A Rad polypeptide has biological activity if it has one, two, three, and preferably more of the following properties: (1) it binds to nm23;; (2) it promotes autophosphorylation of nm23; (3) it has GTP ase activity; (4) it inhibits, competitively or non-competitively, the binding of Rad and nm23. A polypeptide has biological activity if it is an antagonist, agonist, or super-agonist of a polypeptide having one of the above-listed properties.

A nm23 polypeptide has biological activity if it has one, two, three, and preferably more of the following properties: (1) it binds to Rad;; (2) it promotes Rad activity; (3) it has nucleotide diphosphokinase activity; (4) it inhibits, competitively or non-competitively, the binding of Rad and nm23. A polypeptide has biological activity if it is an antagonist, agonist, or super-agonist of a polypeptide having one of the above-listed properties.

“Misexpression”, as used herein, refers to a non-wild type pattern of gene expression. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### DETAILED DESCRIPTION

Rad and nm23 Coregulate Each Other's Activity

Rad (ras-related protein associated with diabetes) is the prototypic member of a new class of GTPasses which includes Rad, Gem and Kir. These three Ras-related GTPases are characterized by N- and C-terminal extensions of 88 and 31 amino acids for Rad, as well as non-conservative changes in primary amino acid sequence as compared to Ras, Rad also lacks a typical prenylation motif, and in cells, Rad is located primarily in the cytosol with some Rad associated with the cytoskeletal. Increased levels of Rad are observed in skeletal muscle of some patients with Type II Diabetes, and Rad expression in cultured muscle and fat cells is associated with inhibition of insulin-stimulated glucose uptake. Rad interacts with several cellular proteins including β-tropomyosin, calmodulin, calmodulin kinase, and nm23. Calmodulin binds to the C-terminal domain of Rad, and binding is inhibited by serine phosphorylation.

By purification and sequencing, the inventors have identified Rad GAP as nm23, a developmental gene in Drosophila and putative tumor metastasis suppressor in humans. nm23 associates with Rad by co-precipitation and promotes GTP hydrolysis, nm23 also promotes GTP reloading through its nucleoside diphosphate kinase activity, thus functionally serving as both GAP and GEF for Rad.

In addition to nm23 regulating Rad activity, Rad appears to regulate nm23 activity. Thus, Rad increases nucleoside diphosphate kinase activity of nm23 to ATP, GTP, CTP, and UTP. Rad also regulates nm23 autophosphorylation. Furthermore, preliminary experiments utilizing dominant/negative forms of Rad, show that Rad may play a role in the effects of nm23 to alter cellular motility, the presumed mechanism by which nm23 affects tumor metastasis potential.

The inventors have discovered a novel bimolecular, bidirectional mechanism of regulation between nm23 and Rad. nm23 regulates Rad's state of activity by altering the balance of GTP and GTP loading, while Rad alters the state of nm23 activity as an NDP kinase. Rad appears to play a role in the pathogenesis of insulin resistance in Type II diabetes, and nm23 appears to play an important role in tumor metastasis in humans and rodents. The invention provides diagnostic and therapeutic agents in the area of diabetes, development and tumorigenesis. Specifically, with respect to diagnostics, the level of Rad expression, as well as nm23 expression, provides a useful marker for tumor metastatic potential. Conversely, nm23 expression, like Rad expression provides a marker for Type II diabetes. From a therapeutic perspective, alterations in Rad activity by nm23 regulation provide a novel mechanism to regulate insulin stimulated glucose transport and/or other components of the insulin action pathway. Again, conversely, Rad provides a tool for regulation of nm23 activity, and a tool for defining agents which regulate nm23 activity, and thus is useful for altering tumor metastatic potential and tumor motility.

Methods of the invention allow manipulation of Rad or nm23 conditioned processes by changing the balance between between Rad and nm23 activity.

Methods of the invention allow the manipulation of Rad levels, by direct manipulation, e.g., promotion, of Rad expression or activity, or by manipulation, e.g., promotion, of nm23 activity or expression, and thereby allow promotion of growth and development, in normal growth and development, and in wound healing in diverse tissues, and particularly in tissues where Rad is expressed, e.g, in heart, lung, and skeletal muscle.

Fragments and Analogs of Rad and nm23

Analogs can differ from naturally occurring proteins in amino acid sequence or in ways that do not involve sequence, or both. Non-sequence modifications include in vivo or in vitro chemical derivatization. Non-sequence modifications include changes in acetylation, methylation, phosphorylation, carboxylation, or glycosylation.

Preferred analogs include Rad and nm23 polypeptides (or biologically active fragments thereof) whose sequences differ from the wild-type sequence by one or more conservative amino acid substitutions or by one or more non-conservative amino acid substitutions, deletions, or insertions which do not abolish the biological activity. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative substitutions can be taken from the table below.

### TABLE 1

### CONSERVATIVE AMINO ACID REPLACEMENTS

| For Amino Acid | Code | Replace with any of |
|---|---|---|
| Alanine | A | D-Ala, Gly, beta-Ala, L-Cys, D-Cys |
| Arginine | R | D-Arg, Lys, D-Lys, homo-Arg, D-homo-Arg, Met, Ile, |
| | | D-Met, D-Ile, Gm, D-Orn |
| Asparagine | N | D-Asn, Asp, D-Asp, Giu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | B | D-Giu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, Met, |
| | | D-Met, Ile, D-Ile, Gm, D-Orn |
| Methionine | M | D-Met, S-Me-Cys, Ile, D-Ile, Leu, D-Leu, Val, D-Val |
| Phenylalanine | F | D-Phe, Tyr, D-Thr, L-Dopa, His, D-His, Tm, D-Trp, |
| | | Trans-3,4, or 5-phenylproline, cis-3,4, or 5-phenylproline |
| Proline | P | D-Pro, L-I-thioazolidine-4-carboxylic acid, D-or L-1-oxazolidine-4- |
| | | carboxyiic acid |
| Serine | S | D-Ser, Thr, D-Thr, allo-Thr, Met, D-Met, Met(O), |
| | | D-Met(O), L-Cys, D-Cys |
| Threonine | T | D-Thr, Ser, D-Ser, allo-Thr, Met, D-Met, Met(O), D- |
| | | Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase peptide stability; such analogs may contain, for example, one or more non-peptide bonds (which replace the peptide bonds) in the peptide sequence. Also included are: analogs that include residues other than naturally occurring L-amino acids, e.g., D-amino acids or non-naturally occurring or synthetic amino acids, e.g., β or γ amino acids; and cyclic analogs.

The nm23 and Rad polypeptides, as used herein, refer to full length gene products and to active fragments and analogs of either of these genes. Rad is described in Zhu et al., 1995, JBC 270: 4805-4812. nm23H1 is described in Stegg et al., 1988, J. Natl. Cancer Inst. 80-200-204. nm23H2 is described in Stahl et al., 1991, Cancer Res 51: 445-449. All references, patents, and patent applications referred to herein are incorporated by reference.

In preferred embodiments the Rad or nm23 polypeptide is a fragment and: the fragment is at least 5, 10, 20, 50, 100, or 150 amino acids in length; the fragment is equal to or less than 200, 150, 100, 50 amino acid residues in length; the fragment is either, an agonist or an antagonist, of a biological activity of a naturally occurring Rad or nm23; the fragment can inhibit, e.g., competitively or noncompetitively inhibit, the binding of Rad to nm23.

In preferred embodiments the fragment or analog of Rad or nm23 has at least 60, and more preferably at least 70, 80, 90, 95, 99, or 100 % sequence identity with a naturally occurring Rad or nm23 sequence.

In a preferred embodiment, the fragment or analog of Rad or nm23 differs in amino acid sequence at up to 1, 2, 3, 5, or 10 residues, from the corresponding residues in a naturally occurring Rad or nm23 sequence. In other preferred embodiments, the fragment differs in amino acid sequence at up to 1, 2, 3, 5, or 10 % of the residues from a naturally occurring Rad or nm23 sequence. In preferred embodiments the differences are such that the fragment exhibits a Rad or nm23 biological activity. In other preferred embodiments the differences are such that the fragment does not have Rad or nm23 biological activity. In preferred embodiments one or more, or all of the differences are conservative amino acid changes. In other preferred embodiments one or more, or all of the differences are other than conservative amino acid changes.

In another aspect, the invention features, a composition which includes a Rad and an nm23 polypeptide (or nucleic acids which encode them) and one or more additional components, e.g., a carrier, diluent, adjuvent, or solvent. The additional component can be one which renders the composition useful for in vitro, in vivo, pharmaceutical, or veterinary use.

Gene Therapy

Gene therapy protocols can be used to deliver nucleic acids encoding either an agonistic or antagonistic form of a Rad or nm23 polypeptide. The invention features expression vectors for in vivo transfection and expression of a Rad or nm23 polypeptides in particular cell types so as to reconstitute the function of, or alternatively, antagonize the function of Rad or nm23 in a cell, e.g., a cell in which that polypeptide is misexpressed. Expression constructs of Rad or nm23 polypeptides, may be administered in any biologically effective carrier, e.g. any formulation or composition capable of effectively delivering the Rad or nm23 encoding sequence to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g. a cDNA, encoding a polypeptide. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol (1992) 158:97-129). It is also one of the few viruses that may integrate its DNA into non-dividing cells, and exhibits a high frequency of stable integration (see for example Flotte et al. (1992) Am. J Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a Rad or nm23 polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes.

In a representative embodiment, a gene encoding a polypeptide can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication W091/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by Stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Antisense Therapy

Another aspect of the invention relates to the use of “antisense” therapy. As used herein, “antisense” therapy refers to administration or in situ generation of oligonucleotides or their derivatives which specifically hybridize under cellular conditions, with the cellular mRNA and/or genomic DNA encoding or controlling the expression of a Rad or nm23 gene so as to inhibit expression of the encoded protein, e.g. by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular MRNA which encodes a protein. Alternatively, the antisense construct is an oligonucleotide probe which is generated ex vivo and which, when introduced into the cell causes inhibition of expression by hybridizing with the mRNA and/or genomic sequences of a Rad or nm23 gene. Such oligonucleotide probes are preferably modified oligonucleotide which are resistant to endogenous nucleases, e.g. exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

Accordingly, the modified oligomers of the invention are useful in therapeutic, diagnostic, and research contexts. In therapeutic applications, the oligomers are utilized in a manner appropriate for antisense therapy in general. For such therapy, the oligomers of the invention can be formulated for a variety of loads of administration, including systemic and topical or localized administration. For systemic administration, injection is preferred, including intramuscular, intravenous, intraperitoneal, and subcutaneous for injection, the oligomers of the invention can be formulated in liquid solutions, preferably in physiologically compatible buffers such as Hank's solution or Ringer's solution. In addition, the oligomers may be formulated in solid form and redissolved or suspended immediately prior to use. Lyophilized forms are also included in the invention.

The compounds can be administered orally, or by transmucosal or transdermal means. For transmucosal or transdermal administration, penetrants appropriate to the barrier to be permeated are used in the formulation. Such penetrants are known in the art, and include, for example, for transmucosal administration bile salts and fusidic acid derivatives, and detergents. Transmucosal administration may be through nasal sprays or using suppositories. For oral administration, the oligomers are formulated into conventional oral administration forms such as capsules, tablets, and tonics. For topical administration, the oligomers of the invention are formulated into ointments, salves, gels, or creams as known in the art.

In addition to use in therapy, the oligomers of the invention may be used as diagnostic reagents to detect the presence or absence of the target DNA or RNA sequences to which they specifically bind.

The antisense constructs of the present invention, by antagonizing the normal biological activity of Rad or nm23, can be used in the manipulation of tissue, both in vivo and in ex vivo tissue cultures.

Transgenic Animals

The invention includes transgenic animals which include cells (of that animal) which contain a Rad and an nm23 transgene. The transgene can encode the wild-type form of the protein, or can encode homologs thereof, including both agonists and antagonists, as well as antisense constructs. In preferred embodiments, the expression of the transgene is restricted to specific subsets of cells, or tissues utilizing, for example, cis-acting sequences that control expression in the desired pattern. Tissue-specific regulatory sequences and conditional regulatory sequences can be used to control expression of the transgene in certain spatial patterns. Temporal patterns of expression can be provided by, for example, conditional recombination systems or prokaryotic transcriptional regulatory sequences. In preferred embodiments, the transgenic animal carries a “knockout” gene.

Genetic techniques which allow for the expression of transgenes, that are regulated in vivo via site-specific genetic manipulation, are known to those skilled in the art. For example, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of the subject polypeptide. For example, excision of a target sequence which interferes with the expression of a recombinant gene, such as one which encodes an antagonistic homolog, can be designed to activate expression of that gene. This interference with expression of the protein can result from a variety of mechanisms, such as spatial separation of the subject gene from the promoter element or an internal stop codon. Moreover, the transgene can be made wherein the coding sequence of the gene is flanked recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

See e.g., descriptions of the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694).

Genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of the recombinant gene can be regulated via control of recombinase expression.

Similar conditional transgenes can be provided using prokaryotic promoter sequences which require prokaryotic proteins to be simultaneous expressed in order to facilitate expression of the transgene. Exemplary promoters and the corresponding trans-activating prokaryotic proteins are given in U.S. Pat. No. 4,833,080. Moreover, expression of the conditional transgenes can be induced by gene therapy-like methods wherein a gene encoding the trans-activating protein, e.g. a recombinase or a prokaryotic protein, is delivered to the tissue and caused to be expressed, such as in a cell-type specific manner. By this method, the transgene could remain silent into adulthood until “turned on” by the introduction of the trans-activator.

Production of Fragments and Analogs

The inventors have discovered an interaction between Rad and nm23. Fragments and analogs of Rad and or nm23 can be used to inhibit or promote this interaction. The sequence of Rad and nm23 are known in the art. One skilled in the art can alter the disclosed structures, e.g., by producing fragments or analogs, and test the newly produced structures for activity. Examples of prior art methods which allow the production and testing of fragments and analogs are discussed below. These, or analogous methods, can be used to make and screen fragments and analogs of Rad, nm23, and other molecules which interact with them.

Generation of Fragments

Fragments of a protein can be produced in several ways, e.g., recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Expression of the mutagenized DNA produces polypeptide fragments. Digestion with “end-nibbling” endonucleases can thus generate DNA's which encode an array of fragments. DNA's which encode fragments of a protein can also be generated by random shearing, restriction digestion or a combination of the above-discussed methods.

Fragments can also be chemically synthesized using techniques known in the art such as conventional Merrifield solid phase f-Moc or t-Boc chemistry. For example, peptides of the present invention may be arbitrarily divided into fragments of desired length with no overlap of the fragments, or divided into overlapping fragments of a desired length.

Generation of Analogs: Production of Altered DNA and Peptide Sequences by Random Methods

Amino acid sequence variants of a protein can be prepared by random mutagenesis of DNA which encodes a protein or a particular domain or region of a protein. Useful methods include PCR mutagenesis and saturation mutagenesis. A library of random amino acid sequence variants can also be generated by the synthesis of a set of degenerate oligonucleotide sequences. (Methods for screening proteins in a library of variants are elsewhere herein.)

PCR Mutagenesis

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn²⁺ to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irRadiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Generation of Analogs: Production of Altered DNA and Peptide Sequences by Directed Mutagenesis

Non-random or directed, mutagenesis techniques can be used to provide specific sequences or mutations in specific regions. These techniques can be used to create variants which include, e.g., deletions, insertions, or substitutions, of residues of the known amino acid sequence of a protein. The sites for mutation can be modified individually or in series, e.g., by (1) substituting first with conserved amino acids and then with more Radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Olionucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. USA, 75: 5765[1978]).

Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. E.g., the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High-Through-Put Methods for Screening Libraries of Peptide Fragments or Homologs

Various techniques are known in the art for screening generated mutant gene products. Techniques for screening large gene libraries often include cloning the gene library into replicable expression vectors, transforming appropriate cells with the resulting library of vectors, and expressing the genes under conditions in which detection of a desired activity, e.g., in this case, binding to a Rad or nm23 polypeptide, or to a downstream intracellular protein, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid assays such as the system described above (as with the other screening methods described herein), can be used to identify fragments or analogs of a Rad or nm23 polypeptide. These may include agonists, superagonists, and antagonists.

Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). In a similar fashion, a detectably labeled ligand can be used to score for potentially functional peptide homologs. Fluorescently labeled ligands, e.g., receptors, can be used to detect homolog which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and fl are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10 ¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Secondary Screens

The high through-put assays described above can be followed by secondary screens in order to identify further biological activities which will, e.g., allow one skilled in the art to differentiate agonists from antagonists. The type of a secondary screen used will depend on the desired activity that needs to be tested. For example, an assay can be developed in which the ability to inhibit an interaction between a protein of interest and its respective ligand can be used to identify antagonists from a group of peptide fragments isolated though one of the primary screens described above.

Therefore, methods for generating fragments and analogs and testing them for activity are known in the art. Once the core sequence of interest is identified, it is routine to perform for one skilled in the art to obtain analogs and fragments.

Peptide Mimetics

The invention also provides for reduction of the protein binding domains of the subject Rad and nm23 polypeptides to generate mimetics, e.g. peptide or non-peptide agents. Preferred peptide mimetics are able to disrupt binding of Rad and nm23. The critical residues of Rad or nm23, can be determined and used to generate Rad or nm23 derived peptidomimetics which competitively or noncompetatively inhibit binding of Rad to nm23 (see, for example, “Peptide inhibitors of human papillomavirus protein binding to retinoblastoma gene protein” European patent applications EP-412,762A and EP-B31,080A). For example, scanning mutagenesis can be used to map the amino acid residues of a particular polypeptide involved in binding a polypeptide, peptidomimetic compounds (e.g. diazepine or isoquinoline derivatives) can be generated which mimic those residues in binding to a counterligand, and which therefore can inhibit binding of the polypeptide to the counterligand.

Non-hydrolyzable peptide analogs of critical residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J. Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J. Chem Soc Perkin Trans 1: 1231), and β-aminoalcohols (Gordon et al. (1985) Biochem Biophys Res Commun 126:419; and Dann et al. (1986) Biochem Biophys Res Commun 134:71).

Drug Screening Assays

The invention provides assays which can be used to screen for drugs which are either agonists or antagonists of the normal cellular function, in this case, of the subject Rad and nm23 polypeptides, or of their role in intracellular signaling. In one embodiment, the assay evaluates the ability of a compound to modulate binding between a Rad polypeptide and a nm23 polypeptide. A variety of assay formats will suffice and, in light of the present inventions, will be comprehended by skilled artisan.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or change in enzymatic properties of the molecular target.

### Other Embodiments

Other embodiments are within the following claims.

What is claimed is:

1. A method of modulating an activity of nm23 in a subject at risk for a proliferative disorder comprising: modulating the level of Rad activity.
